(19) United States
(12) Patent Application Publication
(10) Pub. No.: US 2022/0390400 A1
(43) Pub. Date: Dec. 8, 2022

(54) Ammonia Sensor Including Thermal Indicator Component and Acid-Functional Sorbent, and Method of Use

(71) Applicant: 3M INNOVATIVE PROPERTIES COMPANY, St. Paul, MN (US)

(72) Inventors: Michael S. Wendland, North St. Paul, MN (US); Michael W. Kobe, St. Paul, MN (US); Richard C. Webb, St. Paul, MN (US); Michael E. Hamerly, Vadnais Heights, MN (US)

(21) Appl. No.: 17/708,533

(22) Filed: Mar. 30, 2022

Related U.S. Application Data
(60) Provisional application No. 63/192,732, filed on May 25, 2021.

(51) Int. Cl. G01N 25/48; G01N 21/78

(52) U.S. Cl. CPC G01N 25/4873; G01N 21/783

(57) ABSTRACT

The present disclosure provides an ammonia sensor and method of use. The sensor includes: at least one thermal indicator component independently selected from an electronic thermal sensor, an irreversible temperature indicator, and a heat-shrinkable film; an acid-functional porous sorbent in thermal contact with the at least one thermal indicator component; and an acid having a boiling point above 120° C. and a pKa of no greater than 2.5. The acid is impregnated in or covalently attached to the porous sorbent. The method includes: placing an ammonia sensor in contact with a container holding a volume of ammonia; and monitoring the ammonia sensor for a detectable response from the at least one thermal indicator component due to contact of ammonia with the acid that generates thermal energy sufficient to cause the response.

### BACKGROUND

Ammonia in its various forms is an important industrial chemical. It is used in large volumes in a wide variety of applications with some of the more important applications being agriculture (fertilizer) and industrial refrigeration (coolant). Because of its large volume use, ammonia is stored in very large quantities at production and distribution sites (millions of gallons per tank in liquified form) and transported in various large containers (train cars, semi-trailers). In addition, because it is commonly handled and used as a concentrated gas sometimes in enclosed spaces, there exist ample situations were a leak or accident can result in an environment near the lower flammability limit (160,000 ppm) where ammonia poses an explosion risk. Ammonia is a harmful gas to human health even at relatively low concentrations (e.g., NIOSH IDLH (immediate danger to life and health)=300 ppm). For these reasons, there has been a long ongoing need for sensors and indicators in these application spaces which can detect ammonia over a wide range of concentrations.

Various sensor technologies are currently utilized as ammonia sensors. Electrochemical and polymer thin film capacitive sensors are the most sensitive with electrochemical sensors being the most used sensors for low concentrations of ammonia (single ppm to <1,000 ppm). Metal oxide and infrared (IR) sensors are typically used when trying to detect or alert to ammonia concentrations >1,000 ppm. These sensor technologies have upper detection limits around 20,000-30,000 ppm.

One specific application that requires the ability to detect ammonia over a very wide range of concentrations is industrial refrigeration. Ammonia is a very energy efficient coolant used in large scale refrigeration. However, again because ammonia is a dangerous gas, there are strict regulations in place around ammonia detection. Low concentrations of ammonia (<300 ppm) are important to be able to monitor for worker safety, mid-range concentrations (1,000-10,000 ppm) are important to monitor to trigger alarms and initiate emergency ventilation equipment during instances of significant leaks, and high concentrations (>10,000 ppm) are important to monitor to trigger automatic machine cut-offs for combustion driven compressors and allow emergency response personnel to assess explosion risks (e.g., emergency procedures including evacuation are triggered when an environment reaches an ammonia concentration higher than 40,000 ppm which is 25% the LFL).

Existing ammonia sensor technologies require power and some are not very compact (e.g., IR sensors). For this reason, these sensors are used as area sensors (so they can be plugged in) and lower concentration assessment for worker safety is done with handheld devices. Source points for the ammonia are therefore not very easy to find without someone walking around with a handheld or cart sized device, pausing for a few minutes to take a reading, and then moving on to measure another spot to see in which direction the concentration increases hoping to find the source. Especially in industrial refrigeration, the enclosed space is well ventilated because of the hazardous nature of ammonia, thus further complicating the ability to identify source points of leaks of ammonia. Further, the extensive amount of piping and connections in such equipment makes for a significant number of potential leak points. It is not uncommon for sulfur sticks (stick of smoldering sulfur which has colorless smoke that turns white if ammonia is around) to still be used to help locate low level leaks of ammonia.

### SUMMARY

In a first aspect, the present disclosure provides an ammonia sensor. The sensor comprises: at least one thermal indicator component independently selected from an electronic thermal sensor, an irreversible temperature indicator, or a heat-shrinkable film; an acid-functional porous sorbent in thermal contact (which may or may not be direct physical contact) with the at least one thermal indicator component; and an acid having a boiling point above 120° C. and a pKa of no greater than 2.5, wherein the acid is impregnated in or covalently attached to the porous sorbent.

In a second aspect, the present disclosure provides an array comprising a plurality of the ammonia sensors according to the first aspect.

In a third aspect, the present disclosure provides a method of detecting ammonia. The method comprises: placing an ammonia sensor according to the first aspect in contact with a container holding a volume of ammonia; and monitoring the ammonia sensor for a detectable response from the at least one thermal indicator component due to contact of ammonia with the acid that generates thermal energy sufficient to cause the response.

This sensor utilizes an exothermic interaction between ammonia and the acid in contact with the (high surface area) porous sorbent. The heat generated from the interaction, which is typically reactive (as opposed to catalytic), causes a detectable response from the thermal indicator component(s), for instance a dimensional change in a heat-shrinkable film, an electronic signal (or end to an electronic signal) in an electronic thermal sensor, and/or a colorimetric change to an irreversible temperature indicator. The detectable response from the thermal indicator component(s), as a result of this activation by ammonia, is what indicates the presence of ammonia outside of its container.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### BRIEF DESCRIPTION OF THE DRAWINGS

FIG. 1 is a schematic top view of one embodiment of an ammonia sensor of the present disclosure, wherein FIG. 1a represents the sensor before activation by ammonia and FIG. 1b represents the sensor after activation by ammonia.

FIG. 2 is a cross-section of the ammonia sensor of FIG. 1a.

FIG. 3 is a schematic top view of an alternative embodiment of an ammonia sensor of the present disclosure, wherein FIG. 3a represents the sensor before activation by ammonia and FIG. 3b represents the sensor after activation by ammonia.

FIG. 4 is a cross-section of the ammonia sensor of FIG. 3a.

FIG. 5 is a schematic top view of a physical pack holding another embodiment of an ammonia sensor of the present disclosure.

FIG. 6 is a representation of the ammonia sensor of FIG. 5 before activation by ammonia, wherein FIG. 6a represents a top view and FIG. 6b a cross-sectional view.

FIG. 7 is a representation of the ammonia sensor of FIG. 5 after activation by ammonia, wherein FIG. 7a represents a top view and FIG. 7b a cross-sectional view.

FIG. 8a is a schematic top view of an exemplary ammonia sensor according to the present disclosure.

FIG. 8b is a cross-section of the ammonia sensor of FIG. 8a, along lines 8b-8b.

FIG. 8c is a schematic top view of the ammonia sensor of FIG. 8a after activation by ammonia.

FIG. 9 is a schematic cross-sectional view of another exemplary ammonia sensor according to the present disclosure.

FIG. 10 is a schematic cross-sectional view of an additional exemplary ammonia sensor according to the present disclosure.

FIG. 11 is a schematic cross-section view of a further exemplary ammonia sensor according to the present disclosure.

While the above-identified figures set forth several embodiments of the disclosure, other embodiments are also contemplated, as noted in the description. The figures are not necessarily drawn to scale. In all cases, this disclosure presents the invention by way of representation and not limitation.

### DETAILED DESCRIPTION OF ILLUSTRATIVE EMBODIMENTS

### Glossary

The term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a,” “an,” and “the” are not intended to refer to only a singular entity but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

The term “and/or” means one or all the listed elements or a combination of any two or more of the listed elements. The term “or” is generally employed in its usual sense including “and/or” unless the content clearly dictates otherwise.

Also, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure provides an ammonia sensor that includes one or more thermal indicator component(s) and an acid-functional sorbent. In a first aspect, an ammonia sensor comprises:
at least one thermal indicator component independently selected from an electronic thermal sensor, an irreversible temperature indicator, and a heat-shrinkable film;
an acid-functional porous sorbent in thermal contact with the at least one thermal indicator component; and
an acid having a boiling point above 120° C. and a pKa of no greater than 2.5, wherein the acid is impregnated in or covalently attached to the porous sorbent.

In a second aspect, the present disclosure provides an array comprising a plurality of the ammonia sensors according to any embodiment(s) of the first aspect. The array may include a plurality of the same sensor or any combination of different embodiments of the ammonia sensors described in the first aspect.

In a third aspect, the present disclosure also provides a method of detecting ammonia. The method comprises:
placing an ammonia sensor (according to any embodiment of the first aspect) in contact with a container holding a volume of ammonia; and
monitoring the ammonia sensor for a detectable response from the at least one thermal indicator component due to contact of ammonia with the acid that generates thermal energy sufficient to cause the response.

Optionally, the detecting completion comprises exposing an underlying image.

Each of the first, second, and third aspects are discussed in detail below.

As noted above, ammonia is a hazardous material and sensors have been used to monitor for its presence. Sensors, arrays, and methods according to at least certain embodiments of the present disclosure are suitable for sensing the presence of ammonia that has escaped from proper containment (e.g., leaked from a container). For instance, a sensor can be used directly at and/or adjacent to connection points, where leaks are likely to occur (e.g., at valves, compressors, seams, etc.), in addition to on walls or other surfaces of a container (e.g., tank, pipe, train car, semi-trailer, etc.).

In certain embodiments, the acid-functional porous sorbent is adhered to the thermal indicator component, such as by using an adhesive (e.g., a layer of adhesive). Multiple such ammonia sensors can be incorporated into an array if desired.

This sensor involves an ammonia presence indicator that utilizes an exothermic interaction between ammonia and the acid. The heat generated from the interaction, which is typically reactive, causes a detectable response from the thermal indicator component(s), for instance a dimensional change (i.e., shrinkage) in a heat-shrinkable film, an electronic signal in an electronic thermal sensor, and/or a color change in an irreversible temperature indicator. The detectable response from the thermal indicator component(s), as a result of this activation by ammonia, is what indicates the presence of ammonia in the atmosphere. The degree of the response will depend on the amount of heat to which the thermal indicator component is exposed, which will depend on the amount of ammonia to which the acid-functional sorbent is exposed.

The at least one thermal indicator component is independently selected from the group consisting of an electronic thermal sensor, an irreversible temperature indicator, and a heat-shrinkable film. When an electronic thermal sensor is present it often comprises at least one of a thermocouple, a resistor, a capacitor, an inductor, or an electronic circuit that changes (e.g., the output voltage or current alters, or at least one portion of the circuit fails) when exposed to a specific minimum elevated temperature. Depending on the particular thermal indicator component, any combination of these are included in an electronic thermal sensor. In some cases, the electronic thermal sensor comprises a thermocouple, e.g., an open junction thermocouple. In various embodiments, for instance and without limitation, T-type, J-type, or E-type thermocouples, may be conveniently used. In some cases, the electronic thermal sensor comprises an electronic circuit that fails when exposed to a specific minimum elevated temperature. Often, the specific minimum elevated temperature is 40 degrees Celsius (° C.), 45° C., 50° C., 55° C. or even 60° C. The electronic circuit may comprise a plurality of electric or electronic components, like, for example, electrodes, wires, capacitors, transistors, resistors, inductors, wire coils, or integrated circuits. The electronic circuit may exhibit a detectable change to its operation as a result of exposure to the specific minimum elevated temperature. The electronic circuit may fail, for instance due to expansion and contraction of at least one component of the circuit, burning/melting of at least one circuit component, a substrate (e.g., heat-shrinkable substrate) that deforms, etc. In some cases, the electronic thermal sensor comprises a resistor. Resistors are well known in the art and their resistance changes with temperature, thus acting as temperature sensors. In some cases, the electronic thermal sensor comprises a capacitor. Capacitors are well known in the art and maintain an electric charge between conductive plates. Some capacitors that are used in sensors change their values in response to a stimulus, such as temperature, thus acting as temperature sensors. In some cases, the electronic thermal sensor comprises an inductor. Inductors are well known in the art. Some inductors that are used in sensors change their values in response to a stimulus, such as temperature, thus acting as temperature sensors.

In some cases, the electronic thermal sensor comprises an RFID (radio-frequency identification) tag. Radio-frequency identification uses electromagnetic fields to identify and track tags, such as RFID tags attached to objects. In operation, an RF reader sends out RF signals (e.g., through an antenna) to create an electromagnetic field. The field activates one or more RFID tags, which each produce a response that provides identifying information back to the RFID reader. When exposed to at least a certain minimum temperature, one or more electronic components of the RFID will fail and no longer produce a response, like an electronic circuit that fails, or a substrate (e.g., shrinkable substrate) that deforms, when exposed to a specific minimum elevated temperature. Once the RF reader ceases to receive the response from the RFID tag that has been exposed to heat, the detectable response from the thermal indicator component comprising an RFID tag is obtained. Alternatively, the RFID tag may have at least one component that provides an altered response to the RF reader in response to heat, to provide a detectable response.

When the irreversible temperature indicator is present, it often comprises a thermochromic dye. Suitable indicators having a thermochromic dye include, for instance, single use multiple-point temperature-indicating labels commercially available from McMaster-Carr (Elmhurst, Ill.), which can be attached to an object using the adhesive provided on the back of the label and show incremental temperature changes. Such labels operate by including a plurality of windows on the front face of the labels that (permanently) change color, such as by turning from white to black, when the temperature reaches each identified temperature point. For use herein, an acid-functional porous sorbent is placed in thermal contact with the irreversible temperature indicator, e.g., such as in contact with the adhesive on the back of a temperature-indicating label.

When the heat-shrinkable film is present, the shrinkage of the heat-shrinkable film provides a visual indicator. For example, the original dimensions of the film can simply be noted by an outline around it. Shrinkage of the film results in revealing of the outline. Alternatively, or additionally, shrinkage of the film can reveal an underlying picture, word, colored feature, or differently colored backing substrate.

The sensors of the present disclosure can be tailored by several factors in the construction of the sensor. Such factors include, for example, the sorbent loading in terms of grams sorbent per square meter of an area of the sensor on which the sorbent is disposed, the identity of the acid, the concentration of acid in terms of millimoles per gram or weight percent per gram of acid-functional porous sorbent, and the type of thermal indicator component. When the acid-functional porous sorbent is adhered to the thermal indicator component using a layer of an adhesive, the layer of adhesive can function as a buffer or insulator. Thus, the thickness of the adhesive can be tailored to control the extent of thermal response from the thermal indicator component.

In certain embodiments, a plurality of ammonia sensors of the present disclosure may be included in an array. In such an array, each of the ammonia sensors may respond differently to ammonia. The sensors in the array may vary with respect to the concentration of acid in the sorbent, the identity of the acid, type of thermal indicator component, and/or the thickness of an (optional) adhesive. Using this variability between sensors of an array can be utilized, for example, to indicate different levels of ammonia.

The heat generated from the interaction of ammonia and the acid causes a detectable response from the thermal indicator component(s), for instance a dimensional change (i.e., shrinkage) in a heat-shrinkable film, an electronic signal in an electronic thermal sensor, and/or a color change in an irreversible temperature indicator. The detectable response from the thermal indicator component(s), as a result of this activation by ammonia, is what indicates the presence of ammonia outside of a container (e.g., tank, pipe, train car, semi-trailer, etc.) holding the ammonia. The degree of response will depend on the amount of heat generated, which is dependent on the amount of ammonia to which the acid-functional porous sorbent was exposed.

In some cases, an ammonia sensor detects ammonia at a concentration of 300 parts per million (ppm) or greater in a gas (e.g., the atmosphere adjacent to an ammonia container), such as 500 ppm or greater, 1,000 ppm or greater, 5,000 ppm or greater, 10,000 ppm or greater, 25,000 ppm or greater, 50,000 ppm or greater, 75,000 ppm or greater, or 100,000 ppm or greater; and 200,000 ppm or less ammonia in a gas.

In certain embodiments, the sensor indicates the presence of ammonia by exposing an underlying visual indicator (e.g., image). Alternatively, the sensor indicates the presence of ammonia by creating a visual indicator on the film. In certain embodiments, the sensor indicates the presence of ammonia by producing a detectable response from an electronic thermal sensor.

In certain embodiments, shrinkage in one dimension of a heat-shrinkable film is at least 5%, at least 8%, or at least 10%. In certain embodiments, shrinkage in the total area of a heat-shrinkable film is at least 10%, at least 15%, or at least 18%. In certain embodiments, shrinkage of the film results in separating or tearing perforations in the heat-shrinkable film.

### Acid-Functional Porous Sorbent

A porous sorbent in thermal contact with the thermal indicator component is also in contact with an acid. In this context, an “acid” includes acid compounds as well as acid moieties. The porous sorbent functions as a scaffold for one or more acids, whether such acid is impregnated therein or covalently attached thereto. Hence, such sorbent is referred to herein as an “acid-functional porous sorbent” when it includes covalently attached acid moieties or impregnated acid compounds. The acid-functional porous sorbent is derived from a porous (e.g., microporous, mesoporous, or macroporous) sorbent material.

Suitable acids include acid compounds or acid moieties having a boiling point above 120° C. and a pKa of no greater than 2.5. Examples of acid compounds that may be incorporated into the porous sorbent include trichloroacetic acid, sulfuric acid, phosphoric acid, alkyl sulfonic acid (e.g., methanesulfonic acid), alkyl phosphonic acid (e.g., methanephosphonic acid), benzene sulfonic acid, and toluene sulfonic acid. These acids may be used individually or in any combination. In this context, “alkyl” refers to C1-C4 alkyl groups, with methyl and ethyl being preferred. Examples of acid moieties (i.e., acid groups) that may be covalently attached to the porous sorbent include sulfonic acid (—SO₃H) and phosphonic acid (—PO₃H₂) groups. Various combinations of acid compounds and acid moieties may be used as desired.

In certain embodiments, the acid-functional porous sorbent is present in an amount of at least 10 grams per square meter (gsm), at least 20 gsm, or at least 40 gsm, of an area of the sensor on which the sorbent is disposed. In certain embodiments, the acid-functional porous sorbent is present in an amount of up to 1000 gsm, up to 600 gsm, up to 400 gsm, or up to 250 gsm, of an area of the sensor on which the sorbent is disposed.

In certain embodiments, if the acid is in the form of acid moieties (i.e., acid groups) that are covalently attached to the porous sorbent to form the acid-functional porous sorbent, they are present in an amount of at least 0.5 millimole acid moieties per gram of acid-functional porous sorbent (mmole/g), at least 1 mmole/g, at least 1.5 mmole/g, at least 2 mmole/g, at least 2.5 mmole/g, or at least 3 mmole/g (millimoles of acid moieties per gram of acid-functional porous sorbent); and up to 5.5 mmole of acid moieties per gram of acid-functional porous sorbent, up to 5 mmole/g, or up to 4.5 mmole of acid moieties per gram of acid-functional porous sorbent.

In certain embodiments, if the acid is an acid compound impregnated in the porous sorbent to form the acid-functional porous sorbent, it is present in an amount of at least 5 percent by weight (wt. %), at least 10 wt. %, or at least 20 wt. % based on the total weight of the acid-functional porous sorbent. In certain embodiments, if the acid is an acid compound impregnated in the porous sorbent to form the acid-functional porous sorbent, it is present in an amount of up to 80 wt. %, up to 70 wt. %, or up to 60 wt. %, based on the total weight of the acid-functional porous sorbent.

The “porous” sorbent includes minute spaces or holes through which liquid or air may pass. It may include a microporous, mesoporous, or macroporous material. A mesoporous material is a material having pores with diameters of 2 nanometers to 50 nm, a microporous material is a material having pores smaller than 2 nm in diameter, and a macroporous material is a material having pores larger than 50 nm in diameter. The amount of nitrogen gas absorbed by the porous sorbent under cryogenic conditions at a relative pressure of 0.98 may be used to measure the total pore volume for pores having diameters up to 50 nanometers. This method measures both micropores and mesopores. The pore volume of the porous sorbent at a relative pressure of 0.98 is often at least 0.2 cm³/gram, at least 0.4 cm³/gram, at least 0.6 cm³/gram, at least 0.8 cm³/gram, at least 1.0 cm³/gram, or at least 1.2 cm³/gram; and no more than 2.5 cm³/gram. In some embodiments, the pore volume of the porous sorbent is substantially macroporous and has quite low microporosity and mesoporosity.

Porous sorbents include those that can withstand the acids described herein without degradation. They may include inorganic materials, organic materials, or combinations thereof. Examples include activated carbon, porous silica, and porous organic polymers. Either individual porous sorbents or any combination of porous sorbents may be used if desired.

In certain embodiments, the porous sorbents include porous organic polymers. In certain embodiments, the porous organic polymers are derived from at least 60 wt. % aromatic monomers. Exemplary porous organic polymers include styrene-divinylbenzene, divinylbenzene-maleic anhydride (such as those described in International Publication No. WO 2017/106434 (3M Innovative Properties Co.)), and other aromatic-containing polymers. Combinations of porous sorbents may be used if desired. In certain embodiments, the porous organic polymers may be functionalized with sulfonic acid groups or other acid moieties as described herein. A precursor polymeric material (one type of porous sorbent material) that is reacted with an agent that provides such groups (e.g., an acid functionalizing agent such as a sulfonic acid agent) to form the acid-functional porous sorbent is typically formed from a polymerizable composition that contains aromatic monomers. Examples of aromatic monomers include, but are not limited to, styrene, styrene substituted with an alkyl group, divinylbenzene (DVB), and the like. Other examples of aromatic monomers include styrene substituted with a chloromethyl group (e.g., vinylbenzyl chloride). Other examples of aromatic monomers include bis(chloromethyl)-substituted aromatic monomers (e.g., p-xylylene-dichloride or isomers thereof). In some embodiments, at least 60 wt. %, at least 70 wt. %, at least 80 wt. %, at least 85 wt. %, at least 90 wt. %, or at least 95 wt. %, and up to 100 wt. %, up to 99 wt. %, up to 98 wt. %, up to 95 wt. %, or up to 90 wt. % of the monomers in the polymerizable composition are aromatic monomers.

The precursor polymeric material is typically crosslinked, thereby forming one type of porous sorbent. Crosslinking tends to enhance the porosity of the precursor polymeric materials and the resulting acid-functional porous sorbent. Any crosslinking method can be used. For example, in some embodiments, the polymerizable composition contains relatively large amounts of DVB (e.g., at least 10 wt. %) and no post-polymerization crosslinking is used. In other embodiments, the precursor is crosslinked lightly by the addition of relatively low amounts of DVB (e.g., 1 wt. % to less than 10 wt. %) in the polymerizable composition. Further crosslinking occurs post-polymerization using Friedel-Crafts chemistry in the presence of compounds having chloromethyl groups or chloromethylether groups such as, for example, xylylene-dichloride (XDC), 1,4-bischloromethyldiphenyl (CMDP), monochlorodimethylether (MCDE), tris-(chloromethyl)-mesitylene (CMM), and p,p′-bis-chloromethyl-1,4-diphenylbutane (DPB). In still other embodiments, the precursor polymeric material can be crosslinked using predominately Friedel-Crafts chemistry by reacting with a linear polymer such as polystyrene. The crosslinking of linear polymers such as polystyrene using Friedel-Crafts chemistry is described, for example, in the reference V. A. Davankov et al., Reactive Polymers, 13, 27-42 (1990). A small molecule crosslinker having chloromethyl or chloromethyl ether groups is added. This reference also describes the post-crosslinking of gel-type polymers (e.g., lightly crosslinked polymers such as polystyrene crosslinked with low amounts of DVB such as 1 wt. %) using Friedel-Crafts chemistry.

Alternatively, gel-type polymers can be prepared from a monomer mixture of styrene and vinylbenzyl chloride (VBC) that is crosslinked with low amounts of DVB such as 2 wt. % or less. The gel-type polymers can be crosslinked using Friedel-Crafts chemistry but no small molecule crosslinker having chloromethyl or chloromethyl ether groups is required. Rather, the chloromethyl group of VBC serves as the crosslinking point. That is, the crosslinker is already part of the gel-type polymer. This method is further described in the reference Jou-Hyeon Ahn et al., Macromolecules, 627-632 (2006).

Macroporous precursor polymeric materials can be prepared from a mixture of styrene and DVB by suspension, emulsion, or precipitation polymerization methods. In precipitation polymerization processes, styrene and DVB monomer mixtures are polymerized in the presence of various solvents that serve as porogens. This can also be accomplished using an emulsion or suspension polymerization method where the organic phase consists of the monomers and the porogen. With precipitation polymerization processes, the reaction product is a monolith that matches the size and shape of the container used for the polymerization reaction. With the emulsion or suspension processes, however, the final polymeric material is typically in the form of beads (particles). The porosity of the resulting polymeric material can be controlled by selection of the amount and identity of the porogen used, the solids content of the polymerization mixture (organic phase), and the amount of crosslinker (e.g., DVB) that is used.

These polymerization methods are further discussed in references such as the following: M. M. Mohamed et al., Nanomaterials, 2, 163-186 (2012); Y. Zhang et al., Nano Today, 4, 135-142 (2009); S. Wei et al., Colloids and Surfaces A: Physiochem. Eng. Aspects, 414, 327-332 (2012); A. K. Nyhus et al., J. Poly. Sci. Part A: Polymer Chemistry, 37, 3973-3990 (1999); Q. Liu et al., J. Phys. Chem. C, 112, 13171-13174 (2008); U.S. Pat. No. 3,531,463 (Gustafson et al.), and U.S. Pat. No. 6,416,487 B1 (Braverman et al.).

Other macroporous precursor polymeric materials can be formed using suspension or emulsion polymerization methods like those described above but with lower levels of the DVB crosslinker (e.g., 2 wt. % to 20 wt. %). A portion of the styrene monomers is replaced with VBC. The resulting polymers can be further crosslinked using Friedel-Crafts chemistry. This method is further described in the reference Jou-Hyeon Ahn et al, Macromolecules, 627-632 (2006).

Still other precursor polymeric materials can be prepared as discussed in the reference C. D. Wood et al., Chem. Mater., 19, 2034-2048 (2007). In this instance, bis(chloromethyl) aromatic monomers such as p-xylylene-dichloride (XDC) or isomers thereof, 4,4′-bis(chloromethyl)-1,1′-biphenyl, and bis(chloromethyl) anthracene are reacted alone or in combination using Friedel-Crafts chemistry to produce micro-, meso-, and macroporous precursor polymeric materials for use as porous sorbents.

Alternatively, precursor polymeric materials can be prepared using even simpler aromatic compounds such as benzene and crosslinking the benzene using Friedel-Crafts chemistry and a small molecule crosslinker such as formaldehyde dimethyl acetal as described in B. Li et al., Macromolecules, 44, 2410-2414 (2011).

In some embodiments, the precursor polymeric material is formed from a polymerizable composition that contains 10 wt. % to 80 wt. % DVB and 20 wt. % to 90 wt. % styrene-type monomers (i.e., styrene and/or styrene substituted with an alkyl group) based on the total weight of monomers in the polymerizable composition. In many such embodiments, at least 90 wt. %, at least 92 wt. %, at least 95 wt. %, at least 96 wt. %, at least 98 wt. %, at least 99 wt. %, and up to 100 wt. % of the monomers in the polymerizable composition are selected from DVB or a styrene-type monomer. In some embodiments, the amount of DVB is at least 10 wt. %, at least 20 wt. %, at least 30 wt. %, at least 40 wt. %, at least 50 wt. %, at least 60 wt. %, and up to 80 wt. %, up to 70 wt. %, up to 60 wt. %, up to 50 wt. %, or up to 40 wt. %, based on the total weight of monomers in the polymerizable composition. The remainder of the monomers is often a styrene-type monomer.

The precursor polymeric material (and other porous sorbents) is typically porous and often has a BET specific surface area that is in a range of 25 square meters per gram (m²/g) to 5000 m²/g. The BET specific surface area is often at least 25 m²/g, at least 50 m²/g, at least 100 m²/g, at least 200 m²/g, at least 300 m²/g, at least 400 m²/g, or at least 500 m²/g, and up to 5000 m²/g, up to 3000 m²/g, up to 1500 m²/g, up to 1200 m²/g, up to 1100 m²/g, up to 1000 m²/g, 5 up to 900 m²/g, up to 800 m²/g, up to 700 m²/g, up to 600 m²/g, or up to 500 m²/g. In some embodiments, BET specific surface area is in a range of 100 m²/g to 5000 m²/g, or in a range of 200 m²/g to 1000 m²/g. Such surface areas also apply to the acid-functional porous sorbents.

In certain embodiments, the precursor polymeric material is treated with an acid-functionalizing agent (e.g., sulfonic acid agent) to form an acid-functional polymeric material (a type of acid-functional porous sorbent). The acid group (e.g., sulfonic acid group (—SO₃H)) typically replaces a hydrogen atom that is bonded to a carbon atom that is part of an aromatic ring of the precursor polymer. Any known method can be used to introduce the acid group into the precursor polymeric material.

In some embodiments, the precursor polymeric material is reacted with a halogenated acid, such as a halogenated sulfonic acid (e.g., chlorosulfonic acid) acid-functionalizing agent. The precursor polymeric material is mixed with a solution of the halogenated acid dissolved in an appropriate organic solvent. Suitable organic solvents include various halogenated solvents such as 1,2-dichloroethane, methylene chloride, and chloroform. The precursor polymeric material is often added to the solution of the halogenated acid at a temperature below room temperature. The initial reaction can be quite exothermic so, if adequate care is not taken, the solvent can boil during the addition. After the reactants are combined, the temperature is often increased to any desired temperature such as room temperature up to the temperature associated with reflux conditions. The reaction time can range from a few minutes to 24 hours. After this reaction, the resulting intermediate polymeric material has attached halogenated acid-functional groups (e.g., —SO₂X groups where X is halo such as chloro). The reaction time and the reaction temperature can be varied to prepare polymeric materials with different amounts of the acid (sulfonyl-containing) group.

To prepare the acid moiety (i.e., acid group), such as sulfonic acid group (—SO₃H), the intermediate polymeric material with attached halogenated acid-functional group (e.g., —SO₂X group) is placed in water. The conversion of the halogenated groups to the acid groups often can occur at room temperature within 30 minutes, within 1 hour, within 2 hours, within 4 hours, within 8 hours, within 12 hours, within 24 hours, within 36 hours, within 48 hours, within 60 hours, or within 72 hours.

In other embodiments, the precursor polymeric material is reacted with concentrated sulfuric acid or with concentrated sulfuric acid in the presence of a catalyst such as silver sulfate. When a catalyst is present, the reaction typically proceeds faster. With or without the catalyst, the reaction temperature is often in a range of room temperature (e.g., 20-25° C.) to 150° C., in a range of room temperature to 125° C., or in a range of room temperature to 100° C. The reaction times can vary from a few minutes (e.g., 5 minutes, 10 minutes, or 30 minutes) to 24 hours or longer. As with halogenated sulfonic acid, the reaction time and the reaction temperature can be varied to prepare polymeric materials with different amounts of the sulfonic acid group. After this reaction, the resulting polymeric material has attached acid groups (e.g., —SO₃H groups), thereby forming an acid-functional porous sorbent.

In certain embodiments, the acid-functional porous sorbent is encapsulated. To keep the acid-functional porous sorbent encapsulated, it may be combined with a binder and formed into a monolith or into a composite particle. The monolith or composite particle is placed in thermal contact with the thermal indicator component, thereby causing the acid-functional porous sorbent to be in thermal contact with the thermal indicator component. The composite construction can be placed in a pouch, for example, that includes holes for passage of the ammonia. Alternatively, it can be placed in a container (e.g., physical pack) that defines a tortuous channel for passage of the ammonia.

### Heat-Shrinkable Film

Useful heat-shrinkable films (i.e., polymer sheets) are also known as shape-memory films (i.e., polymer sheets). Useful heat-shrinkable films may include physically and/or chemically crosslinked polymers.

Suitable physically crosslinked films include linear block copolymers such as thermoplastic polyurethane elastomers with hard segments and soft switching segments. Multi-block copolymers can also serve as films such as, for example, polyurethanes with polystyrene and poly(1,4-butadiene) blocks; ABA tri-block copolymers of poly(tetrahydrofuran) and poly(2-methyl-2-oxazoline); polyhedral oligomeric silsesquioxane (POSS)-modified polynorbornene; and polyethylene/Nylon-6 graft copolymers.

Suitable chemically crosslinked films include, but are not limited to, crosslinked high density polyethylene, crosslinked low-density polyethylene, and crosslinked copolymers of ethylene and vinyl acetate.

Other examples of heat-shrinkable films include polymers selected from polyurethanes, polynorbornenes, polyethers, polyacrylates, polyamides, polysiloxanes, polyether amides, polyether esters, trans-polyisoprenes, polymethyl methacrylates, crosslinked trans-polyoctylenes, crosslinked polyethylenes, crosslinked polycyclooctenes, inorganic-organic hybrid polymers, copolymer blends with polyethylene and styrene-butadiene co-polymers, urethane-butadiene copolymers, polymethyl methacrylate, polycaprolactone, and oligocaprolactone copolymers.

Suitable heat-shrinkable films include polymers such as those described in U.S. Pat. No. 5,506,300 (Ward et al); U.S. Pat. No. 5,145,935 (Hayashi); U.S. Pat. No. 5,665,822 (Bitler et al); U.S. Pat. No. 6,160,084 (Langer); U.S. Pat. No. 6,388,043 (Langer); U.S. Pat. No. 5,155,199 (Hayashi); U.S. Pat. No. 7,173,096 (Mather et al.); U.S. Pat. No. 4,436,858 (Klosiewicz); U.S. Pat. No. 6,423,421 (Banaszak); and U.S. Pat. Appl. Publ. Nos. 2005/244353 (Lendlein et al), U.S. 2007/009465 (Lendlein et al), and 2006/041089 (Mather et al).

Heat-shrinkable polymer films (sheets or rolls) can be processed by heating them to near or above the heat-shrinkable (i.e., shape-memory) transition temperature range of the particular material utilized, then orienting the sheet by stretching or tentoring it in at least one direction (typically down-web when a roll-to-roll process is used) followed by cooling the sheet to lock in the strain caused by the stretching. In some embodiments, the sheet can be oriented in two or more directions. For example, biaxially oriented films can be made by simultaneous down-web and cross-web stretching of the polymer film near or above its transition temperature range followed by cooling. Biaxially oriented films or sheets can have a maximum shrink tension in one direction.

The heat-shrinkable films of the sensors of the present disclosure reach a temperature at or above that which the shrink tension of the heat-shrinkable polymer is sufficiently high to cause a substantial change in one or more dimensions of the sheet. The process of making and orienting heat-shrinkable polymer sheets is well known to those having ordinary skill in the art.

In certain embodiments, the sensors of the present disclosure include a heat-shrinkable film with an area having a strained temporary shape and include at least one of a plurality of perforations having a width therein and a total length. When heated to or above a transition temperature range, the heat-shrinkable polymer sheet at least partially converts from its strained temporary shape to its intrinsic shape. The intrinsic shape of the heat-shrinkable film is the shape to which it returns after the polymer is heated to or above a transition temperature range.

In certain embodiments, it is possible to anneal some heat-shrinkable polymers by heating them to a temperature close to but below the transition temperature range. Depending upon the composition of the polymer, such annealing can cause the temporary shape of the heat-shrinkable polymer to change and substantially eliminate the potential for small changes in shape at temperatures below the shape memory transition temperature range.

Examples of commercially available thermoplastic films include: polyurethanes available under the trade designation DIARY, including the MM, MP, MS, and MB (microbead powder) types series available from SMP Technologies, Inc. of Tokyo, Japan; elastic memory composites available under the trade designation EMC from Composite Technology Development, Inc. of Lafayette, Colo.; and polymers available under the trade designation VERIFLEX from Cornerstone Research Group, Inc. of Dayton, Ohio. The shape memory properties of acrylonitrile-butadiene-styrene (ABS) copolymers, polycarbonate, and polyethylene terephthalate are also disclosed by Hussein et al., in “New Technologies for Active Disassembly: Using the Shape Memory Effect in Engineering Polymers, J. Product Development, 6, 431-449 (2008).

Additional examples of commercially available heat-shrinkable films that can be converted into various shapes include those heat-shrinkable films available under the trade designations CORTUFF, CRYOVAC, and OPTI from Sealed Air Inc. of Elmwood Park, N.J. Additional examples include heat-shrinkable films available under the trade designations SHRINKBOX, VHG, EZ, AFG, ABL and PLAnet from Bemis Clysar of Oshkosh, Wis.

### Optional Adhesive

In certain embodiments, the acid-functional porous sorbent is adhered to the thermal indicator component using a layer of an adhesive. The layer of adhesive may be continuous or discontinuous. It may be laminated to the thermal indicator component or coated thereon (e.g., pattern coated). It may be a double-sided transfer adhesive, a sprayable adhesive, or a hot-melt adhesive.

Exemplary adhesives include those described, for example, in U.S. Pat. No. 7,893,179 (Anderson et al.) and U.S. Pat. No. 9,134,251 (Thomas et al.). For example, the adhesive may include a natural rubber adhesive, a synthetic rubber adhesive, a poly-alpha-olefin adhesive, a styrene block copolymer adhesive, a poly(meth)acrylate adhesive, a silicone adhesive, or mixtures thereof.

The layer of adhesive can function as a buffer or insulator. Thus, the thickness of the adhesive can be tailored to control the extent of thermal response. A typical adhesive layer thickness is at least 1 micron, or at least 25 microns, and often up to 2500 microns, or up to 200 microns.

### Exemplary Ammonia Sensors

Sensors of the present disclosure can be in a variety of shapes and sizes. Typical sensors include a smallest dimension of at least 2.5 centimeters, to avoid getting lost during use.

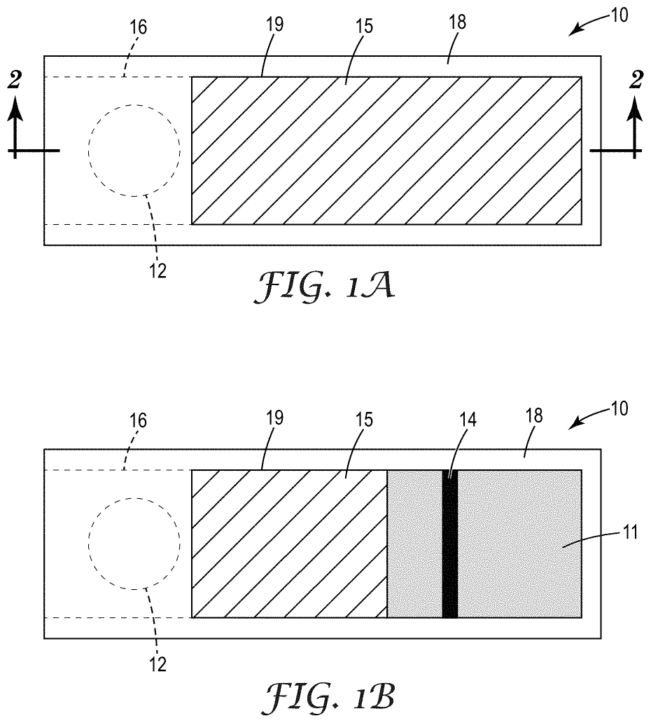

FIGS. 1-11 are representative ammonia sensors.

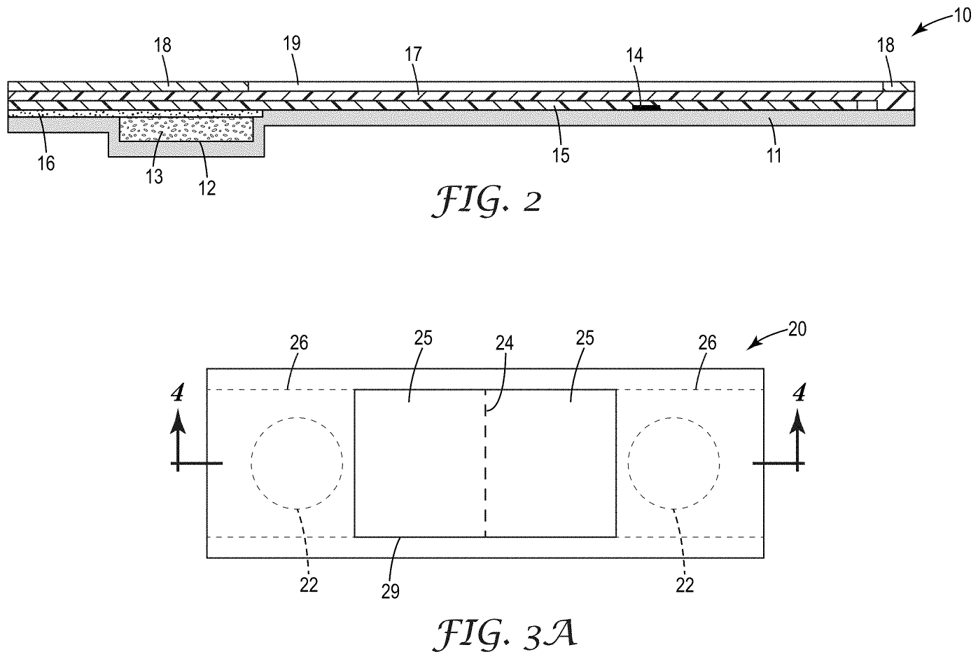

FIG. 1 is a top view of one embodiment of an ammonia sensor of the present disclosure, wherein FIG. 1a represents the sensor before activation by ammonia and FIG. 1b represents the ammonia sensor after activation by ammonia. FIG. 2 is a cross-section of the ammonia sensor of FIG. 1.

Referring to FIGS. 1a, 1b, and 2, the ammonia sensor 10 includes a backing substrate 11 (not shown in FIG. 1a) that includes a reservoir 12 (represented by a dashed line in the form of a circle in FIGS. 1a and 1b) for holding the acid-functional porous sorbent 13 (not shown in FIG. 1a or 1b). Disposed on the backing substrate 11 is visual indicator 14 (e.g., an image such as a picture or word, or a colored feature), such as the black line shown in FIG. 1b. Disposed on the backing substrate 11 (and the visual indicator 14) is a heat-shrinkable film 15, which is adhered to the backing substrate 11 at one end using an adhesive 16 (region of adhesive is represented by a dashed line in the form of a rectangle in FIGS. 1a and 1b). The adhesive 16 locks the heat-shrinkable film 15 in place at one end of the sensor, which causes any shrinkage to move toward the immobilized end in a generally linear fashion. The adhesive 16 also provides thermal contact between the acid-functional porous sorbent 13 and the heat-shrinkable film 15. The ammonia sensor 10 may also include a transparent film 17 (e.g., thermoplastic film not shown in FIGS. 1a and 1b) disposed on the heat-shrinkable film 15, although this is optional. The topmost layer is a cover film 18 (e.g., paper, thermoplastic film) that includes a window 19, through which the heat-shrinkable film 15 is visible. This window 19 may simply be a cut-out creating an opening in the cover film 18.

FIG. 1a shows the heat-shrinkable film 15 prior to detection of the presence of ammonia, which involves an exothermic interaction between ammonia and the acid in the acid-functional porous sorbent. The heat generated from the interaction causes a dimensional change in the heat-shrinkable film 15. The resultant dimensional change (i.e., shrinkage) of the heat-shrinkable film 15 reveals the visual indicator (e.g., a black line) 14 after detection of the ammonia, which is demonstrated by FIG. 1b.

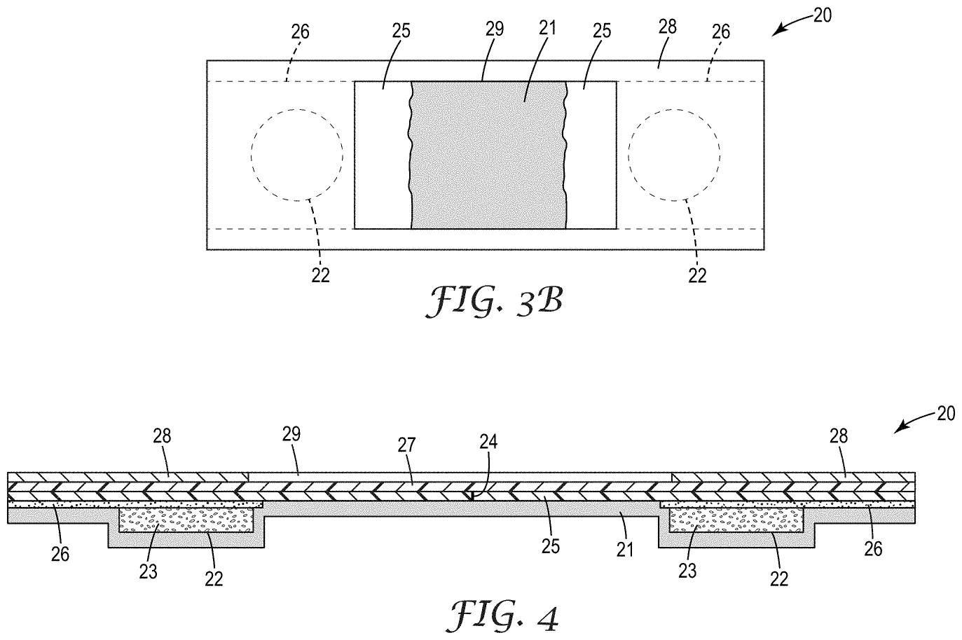

Referring to FIGS. 3a, 3b, and 4, the ammonia sensor 20 includes a backing substrate 21 (not shown in FIG. 3a) that includes two reservoirs 22 (each represented by a dashed line in the form of a circle in FIGS. 3a and 3b) for holding the acid-functional porous sorbent 23 (not shown in FIG. 3a or 3b). Disposed on the backing substrate 21 is a heat-shrinkable film 25 that includes a perforated line 24. The heat-shrinkable film 25 is adhered to the backing substrate 21 at both ends using an adhesive 26 (each region of adhesive represented by a dashed line in the form of a rectangle in FIGS. 3a and 3b). The adhesive 26 locks the heat-shrinkable film 25 in place at both ends of the sensor, which causes any shrinkage to move toward the immobilized ends in a generally linear fashion. The adhesive 26 also provides thermal contact between the acid-functional porous sorbent 23 and the heat-shrinkable film 25. The ammonia sensor 20 may also include a transparent film 27 (e.g., thermoplastic film not shown in FIGS. 3a and 3b) disposed on the heat-shrinkable film 25, although this is optional. The topmost layer is a cover film 28 (e.g., paper, thermoplastic film) that includes a window 29, through which the heat-shrinkable film 25 is visible. This window 29 may simply be a cut-out creating an opening in the cover film 28. Optionally, any ammonia sensor according to the present disclosure may also include a transparent film (e.g., thermoplastic film) disposed on an element that does not generate a significant amount of heat (e.g., a heat-shrinkable film), although this is optional.

FIG. 3a shows the heat-shrinkable film 25 prior to exposure to ammonia, which involves an exothermic interaction between ammonia and the acid in the acid-functional porous sorbent. The heat generated from the interaction causes a dimensional change in the heat-shrinkable film 25. The resultant dimensional change (i.e., shrinkage) of the heat-shrinkable film 25 causes the perforated line 24 to completely perforate and the heat-shrinkable film 25 to separate into two pieces and reveal the backing substrate 21 (which would typically be in a different color for easy visualization) after exposure to ammonia. This is demonstrated by FIG. 3b. The degree of shrinkage and separation between the two pieces of the heat-shrinkable film 25 will depend on the amount of heat to which it is exposed, which will depend on the amount of ammonia to which the acid-functional sorbent is exposed.

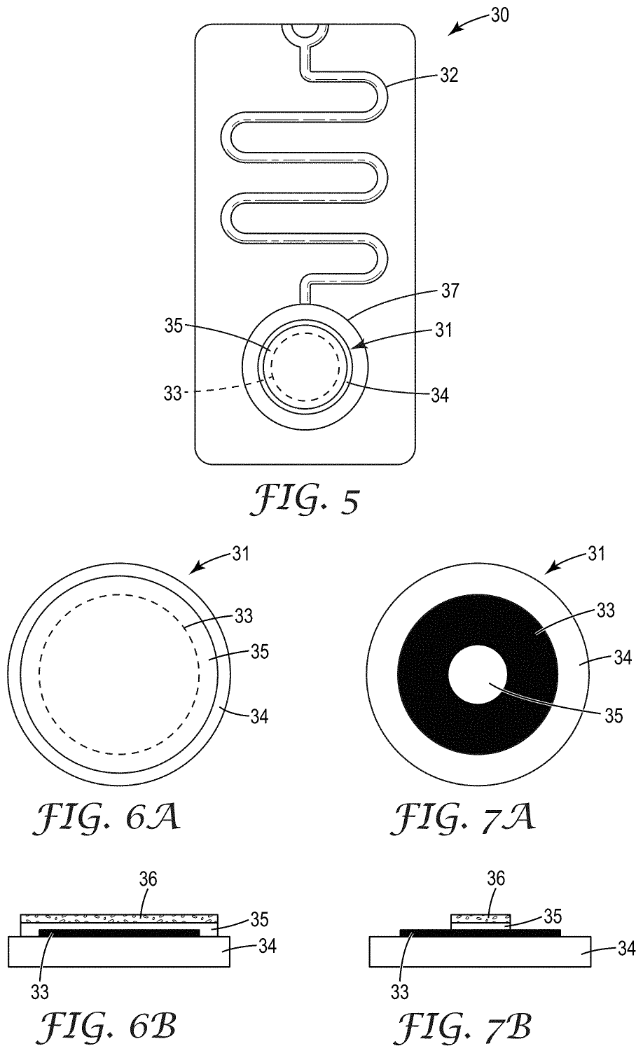

FIG. 5 is a top view of a physical pack holding another embodiment of an ammonia sensor of the present disclosure. The physical pack could include any ammonia sensor described herein. FIG. 6 is a representation of the ammonia sensor of FIG. 5 before activation by ammonia, wherein FIG. 6a represents a top view and FIG. 6b a cross-sectional view. FIG. 7 is a representation of the ammonia sensor of FIG. 5 after activation by ammonia, wherein FIG. 7a represents a top view and FIG. 7b a cross-sectional view. Referring to FIGS. 5, 6a, 6b, 7a, and 7b, the physical pack 30 includes an ammonia sensor 31. The physical pack 30 includes a molded backing (e.g., a thermoplastic backing) that includes a tortuous channel 32 and a reservoir 33, in which is placed the ammonia sensor 31. An (optional) transparent film (e.g., transparent thermoplastic layer) is adhered to the molded backing to allow visualization of the change in the ammonia sensor 31 upon exposure to ammonia. The tortuous channel 32 provides a passage through which the ammonia diffuses through the physical pack 31 into the reservoir 33 where it contacts the ammonia sensor 31. In this embodiment, the ammonia sensor 31 includes a backing substrate 34 on which is disposed a visual indicator 33 (e.g., an image such as a picture or word, or a colored feature), such as the black circle, which represented by the dashed line in FIGS. 5 and 6a and the black circle in FIG. 7a. Covering the visual indicator 33 and disposed on the backing substrate 34 is a heat-shrinkable film 35 that includes acid-functional porous sorbent 36 disposed thereon (and/or incorporated therein). The heat-shrinkable film 35, which is in the form of a circle, is adhered to the backing substrate 34 at the center of the heat-shrinkable film 35. FIGS. 6a and 6b show the heat-shrinkable film 35 prior to exposure to ammonia. The heat generated from the interaction causes a dimensional change in the heat-shrinkable film 35, which forms a smaller circle and reveals the visual indicator 33 after exposure to ammonia. The resultant heat-shrinkable film 35 forms a smaller circle (i.e., a donut hole) as shown FIGS. 7a and 7b. While the ammonia sensor 31 is shown to be circular in this embodiment, any shape could be envisioned. Such a physical pack 30, which modifies the gas or vapor's accessibility to the sensor over time, could be made using sensor components other than a heat-shrinkable film; e.g., the ammonia sensor 31 could include an electronic thermal sensor or irreversible temperature indicator in place of the heat-shrinkable film 35.

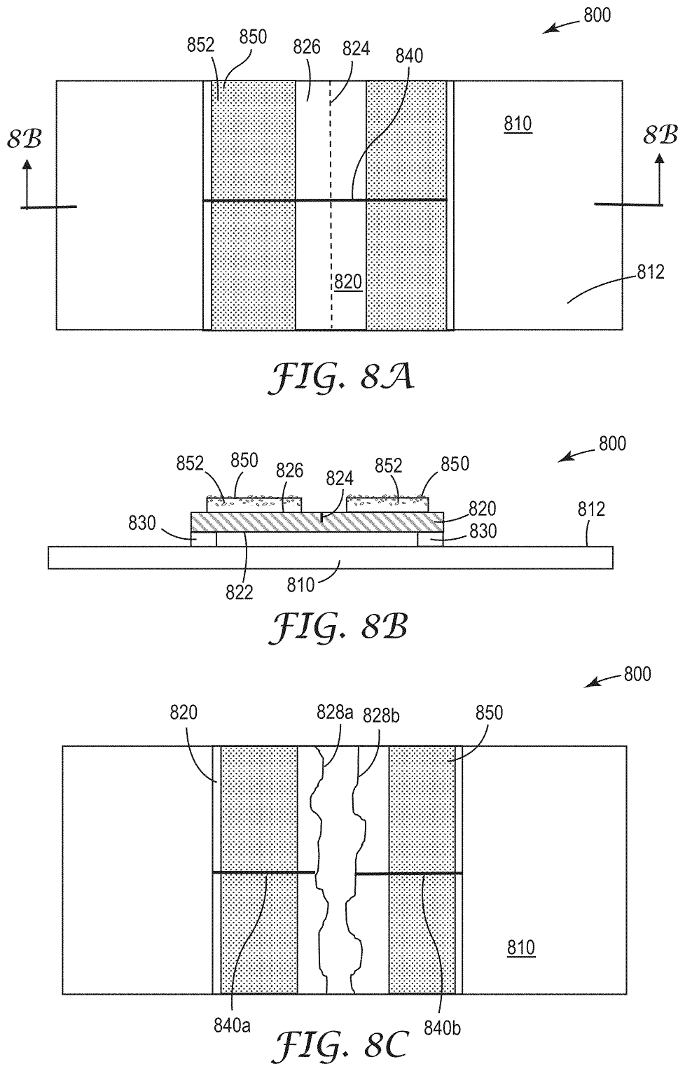

FIG. 8a is a schematic top view of an exemplary ammonia sensor 80 according to the present disclosure, before activation by ammonia. FIG. 8b is a cross-section of the ammonia sensor 80 of FIG. 8a, along lines 8b-8b. Referring to FIGS. 8a and 8b, an ammonia sensor 80 includes a substrate 810 having a major surface 812. A heat-shrinkable film 820 is attached to the major surface 812 of the substrate by an adhesive 830 (not visible in FIG. 8a). In this case, two spaced strips of adhesive 830 are provided, each attached to a first major surface 822 (not visible in FIG. 8a) of the heat-shrinkable film 820 and extending approximately parallel to an opposing edge of the heat-shrinkable film 820. A space is defined between the two lines of adhesive 830 in which the heat-shrinkable film 820 is not adhered to the substrate 810 and the adhesive 830 locks the heat-shrinkable film 820 in place at both ends of the sensor 80, which causes any shrinkage to move toward the immobilized ends in a generally linear fashion. Further, a perforated line 824 is provided down the center of the heat-shrinkable film 820 to provide a point of weakness within the heat-shrinkable film 820. The ammonia sensor 80 also includes a line of a conductive ink 840 (not visible in FIG. 8b) as a thermal indicator component, which is disposed on a second major surface 826 of the heat-shrinkable film 820 orthogonal to the perforated line 824. Additionally, the ammonia sensor 80 includes two spaced strips of adhesive 850, which an acid-functional sorbent 852 is disposed thereon and/or incorporated therein embedded. The adhesive 850 provides thermal contact between the particles of acid-functional sorbent 852 and the heat-shrinkable film 820.

Each strip of adhesive 850 is disposed on the second major surface 826 of the heat-shrinkable film 820 and extending approximately parallel to an opposing edge of the heat-shrinkable film 820.

In operation, the ammonia sensor 80 is exposed to ammonia, and once the acid of the acid-functional sorbent 852 reacts with a sufficient amount of ammonia, the exothermic interaction generates sufficient heat to cause a dimensional change (i.e., shrinkage) in the heat-shrinkable film 820. FIG. 8c is a schematic top view of the ammonia sensor 80 of FIG. 8a after activation by ammonia. More particularly, the ammonia sensor 80 of FIG. 8c shows that sufficient dimensional change (i.e., shrinking) of the heat-shrinkable film 820 results in failure of the heat-shrinkable film 820 at the perforated line 824, splitting the heat-shrinkable film 820 into separate pieces, each having an edge 828a and 828b spaced apart from each other and revealing the backing substrate 810 (which optionally can have a different color than the heat-shrinkable film 820 in at least its center region for easy visualization) after exposure to at least a minimum amount of ammonia. The separation of the heat-shrinkable film 820 into two pieces also separates the line of conductive ink 840 into two partial lines of conductive ink 840a and 840b. The degree of shrinkage and separation between the two pieces of the heat-shrinkable film 820 will depend on the amount of heat to which it is exposed, which is, in part, a function of how much ammonia to which the sensor has been exposed. The ammonia sensor 80 of this embodiment includes the options of both a visible detectable result with the breakage of the heat-shrinkable film 820 into two pieces and an electronic detectable result with the breakage of the line of conductive ink 840 that destroys an electronic circuit that could be connected to the line of conductive ink 840.

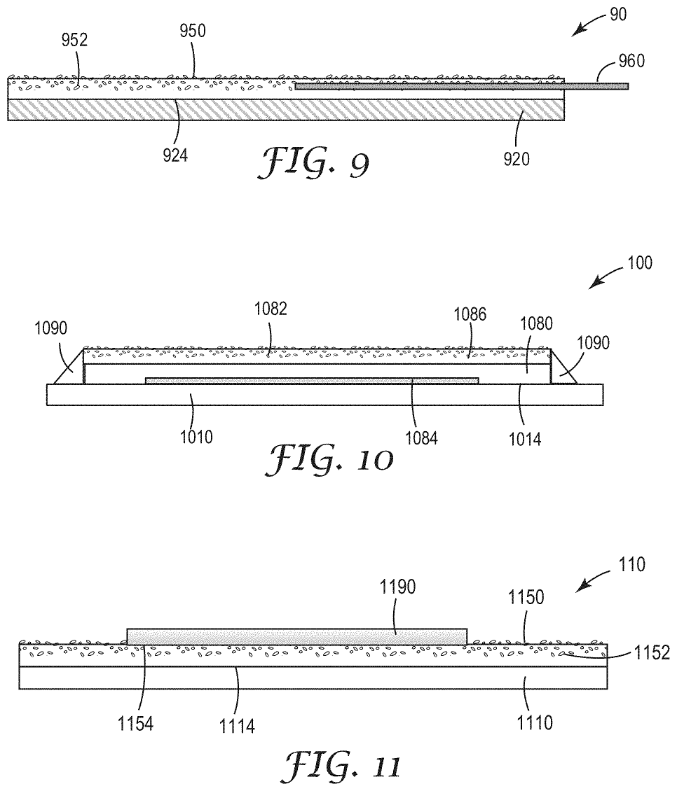

Referring to FIG. 9, a schematic cross-sectional view is provided of another exemplary ammonia sensor 90 according to the present disclosure. The ammonia sensor 90 includes a substrate 920 having a major surface 924. The substrate 920 is not particularly limited, and may be or comprise, for instance, plastic film, glass, thin aluminum film, molded plastic sheet, paper, foil, etc. For sensors that will be applied to a surface that is not flat, a conformable substrate material is preferable. An adhesive 950 is disposed on at least a portion of the major surface 924 of the substrate 920, the adhesive 950 having particles of an acid-functional sorbent 952 embedded within at least an upper portion of the adhesive 950. The ammonia sensor 90 further includes a thermocouple 960 as a thermal indicator component, which is partially embedded in the adhesive 950. Optionally, a second substrate (not shown) could be used with the ammonia sensor 90. For instance, a major surface of the substrate 920 (that is opposite the major surface 924) could be adhered to a second substrate, (e.g., glass, a thin aluminum film, molded plastic sheet, paper, foil, etc.).

In operation, the ammonia sensor 90 is exposed to ammonia, and once the acid of the acid-functional sorbent 952 reacts with a sufficient amount of ammonia, the exothermic interaction generates sufficient heat to cause a detectable response from the thermocouple 960. The skilled practitioner will understand that the thermocouple needs to be attached to some type of electronics to obtain and display temperature measurements from the thermocouple (such as a data acquisition system from Keysight Technologies, Colorado Springs, Colo.).

Referring to FIG. 10, a schematic cross-sectional view is provided of an additional exemplary ammonia sensor 100 according to the present disclosure. The ammonia sensor includes a substrate 1010 having a major surface 1014. An irreversible temperature indicator 1080 is disposed on a major surface 1014 of the substrate 1010. In this embodiment, the irreversible temperature indicator 1080 is adhered to the major surface 1014 of the substrate 1010 using adhesive 1090, on opposing side edges of the irreversible temperature indicator 1080. The irreversible temperature indicator 1080 includes a display region 1084 where a change in temperature can be observed, and the display region 1084 faces the substrate 1010. Preferably, a material is chosen for the substrate 1010 that can be seen through by a user, such as glass or a transparent plastic, although the irreversible temperature indicator 1080 could be removed from the substrate for observation of any temperature change. The irreversible temperature indicator 1080 further includes an adhesive backing 1082 having particles of an acid-functional sorbent 1086 disposed thereon and/or incorporated therein. Optionally, additional adhesive (not shown) may be required to attach the irreversible temperature indicator to an ammonia container.

In operation, the ammonia sensor 100 is exposed to ammonia, and once the acid of the acid-functional sorbent 1086 reacts with a sufficient amount of ammonia, the exothermic interaction generates sufficient heat to cause a detectable response in the irreversible temperature indicator 1080. In some cases, the display region 1084 of the irreversible temperature indicator 1080 comprises a series of areas (e.g., squares, circles, etc.), each labeled with a temperature, and as the heat rises, the areas representing increasingly higher temperatures show a response (e.g., changing from blank (such as white) to filled (such as black).

Referring to FIG. 11, a schematic cross-sectional view is provided of a further exemplary ammonia sensor 110 according to the present disclosure. The ammonia sensor 110 includes a substrate 1110 having a major surface 1114. An adhesive 1150 is disposed on at least a portion of the major surface 1114 of the substrate 1110, the adhesive 1150 having particles of an acid-functional sorbent 1152 disposed thereon and/or incorporated therein. The ammonia sensor 110 further includes an RFID tag 1190 as a thermal indicator component, which is in thermal contact with (e.g., disposed on) a major surface 1154 of the adhesive 1150. In operation, the ammonia sensor 110 is exposed to ammonia, and once the acid of the acid-functional sorbent 1152 reacts with a sufficient amount of ammonia, the exothermic interaction generates sufficient heat to damage the electronic circuitry of the RFID tag 1190. When an RFID reader no longer receives a signal from the RFID tag 1190 or provides an altered response to the RF reader in response to heat to provide a detectable response, the ammonia sensor 110 has indicated the presence of at least a minimum amount of ammonia.

### EXAMPLES

Objects and advantages of this invention are further illustrated by the following examples, but the particular materials and amounts thereof recited in these examples, as well as other conditions and details, should not be construed to unduly limit this invention. These examples are merely for illustrative purposes only and are not meant to be limiting on the scope of the appended claims. Unless otherwise stated, all amounts are in weight percent.

TABLE 1
List of Materials and Suppliers.

| DESIGNATION | DESCRIPTION | SOURCE |
|---|---|---|
| NaOH | Sodium Hydroxide (0.1N aqueous solution) | EMD Millipore Chemicals, |
| | | Billerica, MA |
| Phenolphthalein | Phenolphthalein | Sigma-Aldrich, St. Louis, |
| | | MO |
| NH₄OH | Ammonium Hydroxide (14.5M) | EMD Millipore Chemicals |
| HCl | Hydrochloric Acid (12.1M) | EMD Millipore Chemicals |
| XAD-1180 | Macroporous resin obtained under the trade | Sigma-Aldrich |
| | designation “AMBERLITE XAD-1180” | |
| | (20 × 60 mesh particles) | |
| PAD-910 | Macroporous resin obtained under the trade | Purolite, King of Prussia, |
| | designation “PUROSORB PAD-910” | PA |
| | (20 × 60 mesh particles) | |
| H₂SO₄ | Sulfuric Acid (18M) | EMD Millipore Chemicals |
| Ag₂SO₄ | Silver Sulfate | Alfa Aesar, Ward Hill, MA |
| MeOH | Methanol | EMD Millipore Chemicals |
| Chlorosulfonic acid | Chlorosulfonic acid | Alfa Aesar |
| DCE | 1,2-Dichloroethane | EMD Millipore Chemicals |
| Na₂CO₃ | Sodium Carbonate | VWR International LLC, |
| | | Radnor, PA |
| CH₂Cl₂ | Dichloromethane | EMD Millipore Chemicals |
| K₂CO₃ | Potassium Carbonate | VWR International LLC |
| CER-1 | Cationic exchange resin obtained under the | Alfa Aesar |
| | trade designation “AMBERLYST” 15H (>50 | |
| | mesh/>300 micrometers (μm)) | |
| Activated | Activated Carbon (Kuraray GC, 16 × 35 | Calgon Carbon Corp., |
| Carbon | mesh) | Pittsburgh, PA |
| Silica Gel 60 | Silica Gel 60 (average pore size = 60 Å) | Sigma-Aldrich |
| H₃PO₄ | Phosphoric Acid (14.7M) | EMD Millipore Chemicals |
| Glass slide | Glass micro slide (2 inches × 3 inches; 5.1 | Erie Scientific, Ramsey, |
| | centimeters (cm) × 7.6 cm) | MN |
| Transfer | 5 mil (0.13 millimeters (mm)) 200MP | 3M Company, St. Paul, MN |
| adhesive | transfer adhesive, 2 inches (5.1 cm) wide | |
| Heat-shrink film | Polyethylene heat-shrink film obtained under | Bemis Company, Neenah, |
| | the trade designation “CYLSAR | WI |
| | SHRINKBOX 1525” | |
| Double coated | 6 mil (0.15 mm) 410M double coated tape | 3M Company |
| tape | | |
| Polyester tape | 3M polyester tape 8403 | 3M Company |
| Silver ink | Silver nanoparticle dispersion (50-60 wt. %) | Advanced Nano Products |
| | in tetradecane obtained under the trade | Co., Ltd., Sejong, South |
| | designation “SILVERJET DGH 55LT-25C” | Korea |

### Test Methods

### Gas Sorption Analysis

Porosity and gas adsorption experiments were performed using a Micromeritics Instrument Corporation (Norcross, Ga.) accelerated surface area and porosimetry (ASAP) 2020 system using adsorbates of ultra-high purity. The following is a typical method used for the characterization of the porosity within the exemplified materials. In a Micromeritics half inch diameter sample tube, 50-250 milligrams of material were degassed by heating under ultra-high vacuum (3-7 micrometers Hg) on the analysis port of the ASAP 2020 to remove residual solvent and other adsorbates. The degas procedure for the silica gel, activated carbon, and the XAD-1180 and PAD-910 resins was 3 hours at 150° C. on the degas port followed by 3 hours at 150° C. on the analysis port. The degas procedure for the sulfonated XAD-1180 and PAD-910 resins (PE-1 and PE-2) was 3 hours at 120° C. on the degas port followed by 3 hours at 120° C. on the analysis port. The nitrogen adsorption isotherm at 77 K for each porous material was obtained using low pressure dosing (5 cm³/g) at a relative pressure)(p/p°) less than 0.1 and a pressure table of linearly spaced pressure points from a p/p° in a range from 0.1 to 0.98. The method for the isotherm made use of the following equilibrium intervals: 90 seconds at p/p° less than 10^{-5,} 40 seconds at p/p° in a range of 10^{-5 to} 0.1,and 20 seconds at p/p° greater than 0.1. Helium was used for the free space determination, after nitrogen adsorption analysis, both at ambient temperature and at 77 K. BET specific surface areas (SA_{BET}) were calculated from nitrogen adsorption data by multipoint Brunauer-Emmett-Teller (BET) analysis. Apparent micropore distributions were calculated from nitrogen adsorption data by density functional theory (DFT) analysis using the standard nitrogen at 77 K DFT model. Apparent meso-/macropore distributions were calculated from nitrogen adsorption date by Barrett-Joyner-Halenda (BJH) analysis. Total pore volume was calculated from the total amount of nitrogen adsorbed at a p/p° equal to approximately 0.98. BET, DFT and total pore volume analyses were performed using Micromeritics MicroActive Version 5.02 software.

### Procedure for Determination of Acid Concentration by Liquid Titration

The acid-functional porous sorbent (about 0.200 gram (g)) was suspended in 20 milliliters (mL) of 0.1 N aqueous (aq.) sodium hydroxide (NaOH). To this suspension was added 3 drops of a 1 percent by weight (wt. %) aq. solution of phenolphthalein which caused the suspension to turn a pink color. This suspension was titrated with 0.1 N aq. hydrogen chloride (HCl) until the pink color disappeared and the suspension became clear (phenolphthalein endpoint). The acid concentration was calculated based on the amount of acid needed to reach the titration endpoint, reported as milliequivalents per gram (meq/g).

### Procedure for Exposing Sensors to Ammonia

The sensors were tested for ammonia response using the following procedure. In an 18.5 centimeter (cm) diameter and 10 cm deep (2688 cm³ internal volume) drying dish, 50 mL of 14.5 M ammonium hydroxide (NH₄OH) solution was added, and a glass plate was placed over the drying dish to create an ammonia rich headspace. The small petri dish was inverted to serve as a pedestal to place the indicator sample on such that the indicator sample would be above the NH₄OH solution but not touching the glass plate covering the drying dish.

In the case of sensor samples with heat-shrink film as the thermal indicator component (EX-1 to 6), the sample was visually monitored over an exposure time of 3 minutes. The sample was then removed from the drying dish and the x and y measurements of the indicator sample were measured to calculate the % of shrinkage.

In the case of sensor samples with an irreversible temperature indicator as the thermal indicator component (EX-7 to 11), the sample was exposed to ammonia for 3 minutes. The sample was then removed from the drying dish and visually inspected to determine which sections had changed color and thereby the maximum temperature the sample had reached.

In the case of sensor samples with a thermocouple as the thermal indicator component (EX-12 to 14), the wire of the thermocouple was run out of the ammonia chamber between the top glass plate and the drying dish. The response of the thermocouple was measured and recorded using a Keysight 34972A Data Acquisition Unit.

In the case of the perforated sensor sample (EX-15), the sample was visually monitored over an exposure time of 3 minutes. The sample was then removed from the drying dish and visually inspected to determine if the sample had torn across the perforations and thus severing the silver strips of the sample.

In the case of the RFID based sensor sample (EX-16), the RFID sensor was read real-time by placing the reader (rf IDEAS Wave ID Plus V2 Reader RDR-805W1AKU (rfIDEAS, Rolling Meadows, Ill.) with a typical read range of 1-3 inches (2.5-7.6 cm)) underneath the drying dish. The sample was close enough to the reader sitting within the chamber on its pedestal such that it could be determined when the sample was no longer readable.

### EXAMPLES

### Preparative Example 1 (PE-1): Preparation Of Sulfonated XAD-1180

The following procedure was used to sulfonate the XAD-1180 resin (SA_{BET of} 526.0 m²/gram and a total pore volume of 1.083 cm³/gram (p/p° equal to 0.98)) using silver sulfate (Ag₂SO₄). In a 5 liter (L) round bottom flask equipped with a stir bar, 4.0119 g (12.9 mmol) of Ag₂SO₄ was dissolved in 2.5 L of concentrated (18.0 M) sulfuric acid (H₂SO₄). The round bottom flask was placed in an ice/water bath. To this solution was added slowly 486.26 g of XAD-1180 resin as received (the as received resins are approximately 60% by weight water so the actual amount of resin added was approximately 40 g). The resin instantly turned a reddish/brown color. Once the reaction had cooled down from the addition (approximately 15 minutes), the round bottom flask was stoppered and placed in a sand bath. The reaction mixture was stirred at 100° C. overnight.

After reacting at 100° C. for 18 hours, the reaction was stopped by removing the round bottom flask from the sand bath. The reaction mixture was allowed to cool to room temperature. In a 10 L Erlenmeyer flask, 3.75 L of 6 M aq. H₂SO₄ was prepared by adding 1.25 L of concentrated (18.0 M) H₂SO₄ slowly to 2.5 L of deionized water. The contents of the round bottom flask were slowly poured into the stirred 3.75 L of 6 M aq. H₂SO₄. This mixture was vacuum filtered to isolate the dark red resin particles. The resin particles were washed with ultrapure water (approximately 18 M Ohm resistivity) until the pH of the last 100 mL of the wash was neutral (approximately 10.5 L of water total). The resin particles were washed with 1.5 L of MeOH removing the MeOH by vacuum filtration. The resin particles were placed in a batch oven and dried overnight at 120° C. This sulfonated XAD-1180 resin had a SA_{BET} of 522.4 m²/gram and a total pore volume of 1.242 cm³/gram (p/p° equal to 0.98) as determined by nitrogen adsorption. Titration results showed this sulfonated XAD-1180 resin to have acid equivalents of 2.70 meq/g.

### Preparative Example 2 (PE-2): Preparation of Sulfonated PAD-910

The following procedure was used to sulfonate the PAD-910 resin (SA_{BET} of 618.9 m²/gram and a total pore volume of 1.300 cm³/gram (p/p° equal to 0.98)) using chlorosulfonic acid in 1,2-dichloroethane (DCE). In a 1.0 L round bottom flask, 224.38 g (1.926 moles) of chlorosulfonic acid was poured into 260 mL of DCE while the round bottom flask was cooled in an ice/water bath. To this solution was added 50.26 g of dry PAD-910 resin with the round bottom flask still in the ice/water bath. After mixing for a few minutes, the round bottom flask was removed from the ice/water bath and placed in a sand bath at 90° C. The round bottom flask was equipped with a stir bar and reflux condenser. The reaction was kept under a dry nitrogen atmosphere and reacted at this elevated temperature overnight. The outlet for the nitrogen was bubbled through a saturated aq. sodium carbonate (Na₂CO₃) solution to remove any acidic gas coming from the reaction.

After reacting at 90° C. overnight, the round bottom flask was removed from the sand bath, and the reaction mixture was allowed to cool to room temperature. The reaction mixture was poured into a column (9 cm diameter) using 600 mL CH₂Cl₂ to help transfer. An aq. potassium carbonate (K₂CO₃) solution was prepared by dissolving 500 g of K₂CO₃ in 1000 mL of deionized water. The K₂CO₃ solution was in a 2 L flask, and the flask was placed in an ice/water bath. The solvent from the reaction mixture was slowly dripped into the stirred K₂CO₃ solution. Once the solvent was removed, ultrapure water (approximately 18 M Ohm resistivity) was added using a pipette until the resin particles stopped fuming. The resin particles were washed with ultrapure water (approximately 18 M Ohm resistivity) until the pH of the last 100 mL of the wash was neutral (approximately 16 L of water total). The resin particles were placed in a batch oven and dried overnight at 120° C. This sulfonated PAD-910 resin had a SA_{BET} of 371.0 m²/gram and a total pore volume of 0787 cm³/gram (p/p° equal to 0.98) as determined by nitrogen adsorption. Titration results showed this sulfonated PAD-910 resin to have acid equivalents of 4.3 meq/g.

### Preparative Examples 3-7 (PE-3 TO 7): Preparation of Acid Impregnated Sorbents

Five different acid impregnated sorbents (PE-3 to 7) were prepared using the following procedure. The approximate incipient wet point for each porous sorbent was found by placing approximately 1 g of porous sorbent into a 20 mL vial. Sulfuric acid (18 M) or phosphoric acid (H₃PO₄) (14.7 M) was added to the porous material in 100 or 200 microliter (μL) increments. After each addition of acid, the vial was capped and shook by hand until the liquid was completely within the particles of the porous sorbent. This process of adding acid and then shaking was repeated until liquid was observed to be outside of the particles and staying on the outside even after thorough shaking. The acid impregnated sorbents were prepared using the exact same procedure of incrementally adding acid and shaking, repeating those steps until a total amount of acid was added to the porous sorbent that was just below the incipient wet point volume determined for that porous sorbent.

The porous sorbent used, the impregnant used, the mass of porous sorbent started with and the volume of acid impregnated in the porous sorbent to prepare the acid impregnated sorbents PE-3-7 are summarized in Table 2.

TABLE 2
Details for the preparation of acid
impregnated sorbents (PE-3 to 7).

| | | | Mass of Porous | | |
|---|---|---|---|---|---|
| | Porous | | Material Started | Volume Acid | |
| Sorbent | Material | Impregnant | With, g | Impregnated | |
| PE-3 | Activated | H₂SO₄ | 1.0004 | 700 | μL |
| | Carbon | | | | |
| PE-4 | Silica Gel 60 | H₂SO₄ | 1.0488 | 800 | μL |
| PE-5 | CER-15H | H₂SO₄ | 1.0859 | 400 | μL |
| PE-6 | PE-1 | H₂SO₄ | 1.0832 | 1.6 | mL |
| PE-7 | PE-1 | H₃PO₄ | 1.0247 | 1.5 | mL |

### Examples 1-6 (EX-1 TO 6): Preparation of Heat-Shrink Film Based Sensors

A 2 inch (5.1 cm) wide strip of 200 MP transfer adhesive, with the liner still on one side, was laminated onto CLYSAR Shrinkbox heat-shrink film. A roller was passed over the transfer adhesive several times to ensure complete contact with the heat-shrink film. The laminated material was then cut into a 1.5 inch×1.5 inch (3.8 cm×3.8 cm) square. In one of the corners of the laminated materials, a 1 cm square piece of 410M double coated tape (liner still on one side) was laminated to the heat-shrink film side. A roller was passed over the materials again to ensure proper lamination. The liner was removed from the 1 cm square piece of double coated tape and then the stack was laminated into the center of the 2 inch×3 inch (5.1 cm×7.6 cm) glass slide, with the 200 MP lined transfer adhesive being the top layer. A roller was again passed over the sample to ensure proper lamination of all the layers. A permanent marker was then used to outline the original dimensions of the stack and was used as a reference to measure the degree of shrinkage after ammonia exposure.

The liner on the 200 MP transfer adhesive was then removed to expose the adhesive and allow the sorbent to be bonded to the stack. The sorbent was added by pouring it over the exposed adhesive. To ensure good adhesion of the sorbent, a piece of computer paper was placed over the entire sample and a rubber roller was used to lightly press the sorbent into the adhesive so as to take care to not crush the sorbent particles or break the glass slide. The glass slide was then tipped on its side and lightly tapped to remove any loose sorbent.

Each indicator was exposed to ammonia as described in the Procedure for Exposing Sensors to Ammonia. The sorbent used to prepare each heat-shrink film based sensor sample, the sorbent loading, the measured shrinkage in the x and y direction and the calculated total area shrinkage for EX-1-6 are summarized in Table 3.

TABLE 3
Details of sensor samples EX-1 to 6 and
their response to ammonia exposure.

| | | Sorbent | X-Axis | Y-Axis | Total Area |
|---|---|---|---|---|---|
| | | Loading, | Shrinkage, | Shrinkage, | Shrinkage, |
| Example | Sorbent | gsm* | % | % | % |
| EX-1 | PE-2 | 207 | 8 | 21 | 27 |
| EX-2 | CER-1 | 325 | 29 | 33 | 53 |
| EX-3 | PE-3 | 527 | 50 | 46 | 73 |
| EX-4 | PE-4 | 191 | 42 | 50 | 71 |
| EX-5 | PE-5 | 663 | 58 | 50 | 79 |
| EX-6 | PE-6 | 399 | 38 | 67 | 79 |

*gsm = grams per square meter

### Examples 7-11 (EX-7 TO 11): Preparation of Thermocouple Based Sensors

A 2 inch (5.1 cm) wide strip of 200 MP transfer adhesive, with the liner still on one side, was laminated onto CLYSAR Shrinkbox heat-shrink film. A roller was passed over the transfer adhesive several times to ensure complete contact with the heat-shrink film. The laminated material was then cut into a 1.5 inch×1.5 inch (3.8 cm×3.8 cm) square. In one of the corners of the laminated materials, a 1 cm square piece of 410M double coated tape (liner still on one side) was laminated to the heat-shrink film side. A roller was passed over the materials again to ensure proper lamination. The liner was removed from the 1 cm square piece of double coated tape, and then the stack was laminated into the center of a 2 inch×3 inch (5.1 cm×7.6 cm) glass slide with the 200 MP lined transfer adhesive being the top layer. A roller was again passed over the sample to ensure proper lamination of all the layers.

The liner on the 200 MP transfer adhesive was then removed to expose the adhesive and allow the sorbent to be bonded to the stack. The probe end of a thin wire 40AWG T-type thermocouple was pressed into the exposed adhesive so that the tip of the thermocouple wire was in the center of the sensor. The sorbent was added to the sensor by pouring it over the exposed adhesive. To ensure good adhesion of the sorbent, a piece of weighing paper was placed over the entire sample, and a rubber roller was used to lightly press the sorbent into the adhesive so as to take care to not crush the sorbent particles or break the glass slide. The glass slide was then tipped on its side and lightly tapped to remove any loose sorbent.

Each sensor was exposed to ammonia as described in the Procedure for Exposing Sensors to Ammonia. The sorbent used to prepare each thermocouple based sensor sample, the sorbent loading, the peak temperature measured and the time to reach peak temperature for EX-7-11 are summarized in Table 4.

TABLE 4
Details of sensor samples EX-7 to 11 and
their response to ammonia exposure.

| | | Sorbent | Peak | Time to Peak |
|---|---|---|---|---|
| | | Loading, | Temperature, | Temperature, |
| Example | Sorbent | gsm* | ° C. | seconds |
| EX-7 | CER-1 | 325 | 44 | 200 |
| EX-8 | PE-3 | 67 | 96 | 50 |
| EX-9 | PE-4 | 184 | 94 | 70 |
| EX-10 | PE-6 | 399 | 84 | 75 |
| EX-11 | PE-7 | 212 | 83 | 30 |

*gsm = grams per square meter

### Examples 12-14 (EX-12 TO 14): Preparation of Irreversible Temperature Indicator Based Sensors

3M polyester tape 8403 was placed on the front ends of a Telatemp Temperature Recorder label (Style No. 90859, 38-66° C.) to adhere the label face down on a 2×3 inch (5.1 cm×7.6 cm) glass slide taking care to ensure the tape did not cover any of the indicator sections. The liner on the back side of the label was removed to expose the adhesive and allow the sorbent to be bonded to the back of the label. The sorbent was added by pouring it over the exposed adhesive. To ensure good adhesion of the sorbent, a piece of weighing paper was placed over the entire sample and a rubber roller was used to lightly press the sorbent into the adhesive so as to take care to not crush the sorbent particles or break the glass slide. The glass slide was then tipped on its side and lightly tapped to remove any loose sorbent.

Each sensor sample was exposed to ammonia as described in the Procedure for Exposing Sensors to Ammonia. The sorbent used to prepare each irreversible temperature indicator based sensor sample, the sorbent loading, and the peak temperature indicated on the label for EX-12-14 are summarized in Table 5.

TABLE 5
Details of sensor samples EX-12-14 and
their response to ammonia exposure.

| | | Sorbent | Peak Temperature |
|---|---|---|---|
| Example | Sorbent | Loading, gsm* | Indicated, ° C. |
| EX-12 | CER-1 | 308 | 54 |
| EX-13 | PE-4 | 563 | 66 |
| EX-14 | PE-6 | 383 | 66 |

*gsm = grams per square meter

### Example 15 (EX-15): Preparation of Perforated Circuit Sensor

A perforated sensor sample was prepared by laminating two 0.25 inch×1.5 inch (0.6 cm×3.8 cm) strips of 410M double coated tape to opposite edges of a 1.5 inch×1.5 inch (3.8 cm×3.8 cm) square piece of heat-shrink film. The heat-shrink film was then flipped over, and a line of silver ink (SILVERJET DGH 55LT-25C) was applied to the heat-shrink film using a cotton tipped swab and allowed to thoroughly dry overnight at room temperature for 16 hours. Next, two 0.5 inch×1.5 inch (1.3 cm×3.8 cm) strips of 200 MP lined transfer adhesive were laminated on the same side of the heat-shrink film as the silver ink, 0.2 inch (0.5 cm) from opposite edges of the film, running parallel to the strips of double coated tape. A razor blade was then used to perforate the film by cutting a series of small slits in a line down the center of the film between the two strips of transfer adhesive. The liner of one of the strips of double coated tape was removed and then the exposed adhesive of the tape was applied to the 2 inch×3 inch (5.1 cm×7.6 cm) glass slide. A rubber roller was then used to press the adhesive into the glass slide to ensure proper adhesion. The liner of the other strip of double coated tape was removed and the exposed adhesive of the tape applied to the glass slide so that the heat-shrink film was taut. A rubber roller was then used to press the adhesive into the glass slide and ensure proper adhesion.

The liners of the transfer adhesive were removed to expose the adhesive. The sorbent was added by pouring it over the exposed adhesive. To ensure good adhesion of the sorbent, a piece of weighing paper was placed over the sensor, and then a rubber roller was used to gently press the sorbent into the adhesive. The glass slide was then tipped on its side and lightly tapped to remove any loose sorbent.

A perforated sensor sample (EX-15) was prepared as described above using the sorbent PE-6. This sensor sample had a sorbent loading of 391 gsm. The sensor sample was exposed to ammonia as described in the Procedure for Exposing Sensors to Ammonia. Enough heat was generated during the exposure to activate the heat-shrink film which tore along the perforations, completely disrupting the printed circuit of EX-15.

### Example 16 (EX-16): Preparation of RFID Sensor

A MikroElektronika MIKROE-1475 RFID tag 13.56 MHz (MikroElektronika, Belgrade, Serbia), ISO14443-A was used to prepare a RFID sensor sample (EX-16). This RFID tag was encased in a plastic casing. The top of the casing was removed, and 1.16 g of the sorbent PE-6 was place on the top face of the RFID tag. The sensor sample was exposed to ammonia as described in the Procedure for Exposing Sensors to Ammonia. After exposure for approximately 90 seconds, the reader was no longer able to read the RFID based sensor sample (EX-16). The sensor sample was removed from the drying dish and allowed to stand for several minutes. The reader was still not able to read the RFID based sensor sample (EX-16).

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

What is claimed is:

1. An ammonia sensor comprising:
at least one thermal indicator component independently selected from an electronic thermal sensor, an irreversible temperature indicator, and a heat-shrinkable film;
an acid-functional porous sorbent in thermal contact with the at least one thermal indicator component; and
an acid having a boiling point above 120° C. and a pKa of no greater than 2.5, wherein the acid is impregnated in or covalently attached to the porous sorbent.

2. The ammonia sensor of claim 1, wherein the acid is an acid compound impregnant or a covalently bonded acid moiety,
wherein the acid compound comprises trichloroacetic acid, sulfuric acid, phosphoric acid, methanesulfonic acid, methanephosphonic acid, benzene sulfonic acid, toluene sulfonic acid, or a combination thereof, and
wherein the acid moiety is selected from sulfonic acid (—SO₃H) groups or phosphonic acid (—PO₃H₂) groups.

3. The ammonia sensor of claim 2, wherein the acid compound comprises sulfuric acid, phosphoric acid, methanesulfonic acid, methanephosphonic acid, benzene sulfonic acid, toluene sulfonic acid, or a combination thereof, and wherein the acid moiety is selected from sulfonic acid (—SO₃H) groups or phosphonic acid (—PO₃H₂) groups.

4. The ammonia sensor of claim 1, wherein the electronic thermal sensor is present and comprises at least one of a thermocouple, a resistor, a capacitor, an inductor, or an electronic circuit that changes when exposed to a specific minimum elevated temperature.

5. The ammonia sensor of claim 4, wherein the electronic thermal sensor is present and comprises a thermocouple.

6. The ammonia sensor of claim 3, wherein the electronic thermal sensor is present and comprises an electronic circuit that that changes when exposed to a specific minimum elevated temperature.

7. The ammonia sensor of claim 3, wherein the electronic thermal sensor is present and comprises an RFID tag.

8. The ammonia sensor of claim 3, wherein the electronic thermal sensor is present and comprises at least one of a resistor, a capacitor, or an inductor.

9. The ammonia sensor of claim 1, which detects ammonia at a concentration of 300 parts per million (ppm) or greater in a gas.

10. The ammonia sensor of claim 1, wherein the heat-shrinkable film is present.

11. The ammonia sensor of claim 1, wherein the irreversible temperature indicator is present and comprises a thermochromic dye.

12. The ammonia sensor of claim 1, wherein the acid-functional porous sorbent comprises activated carbon, porous silica, porous organic polymer, or combinations thereof.

13. The ammonia sensor of claim 1, wherein the heat-shrinkable film is present and the acid-functional porous sorbent is present in an amount of 10 gsm to 1000 gsm of an area of the sensor on which the sorbent is disposed.

14. The ammonia sensor of claim 1, wherein the acid comprises acid compounds impregnated within the porous sorbent, wherein the acid is present in an amount of 5 wt. % to 80 wt. %, based on the total weight of the acid-functional porous sorbent.

15. The ammonia sensor of claim 1, wherein the acid comprises acid moieties covalently attached to the porous sorbent, wherein the acid is present in an amount of 0.5 mmole to 5.5 mmole of acid moieties per gram of acid-functional porous sorbent.

16. The ammonia sensor of claim 1, wherein the acid-functional porous sorbent is adhered to the at least one thermal indicator component.

17. An array comprising a plurality of the ammonia sensors of claim 1.

18. A method of detecting ammonia, the method comprising:
placing an ammonia sensor of claim 1 in contact with a container holding a volume of ammonia; and
monitoring the ammonia sensor for a detectable response from the at least one thermal indicator component due to contact of ammonia with the acid that generates thermal energy sufficient to cause the response.

19. The method of detecting of claim 18 wherein detectable response comprises exposing an underlying image.
